(19) United States
(12) Patent Application Publication
(10) Pub. No.: US 2003/0095961 A1
(43) Pub. Date: May 22, 2003

(54) Composition and method for treating disease by increasing activated alpha2 macroglobulin in the blood and extravascular tissue

(76) Inventors: Devin Houston, Siloam Springs, AR (US); John Davidson, Kissee Mills, MO (US); Anthony W. Collier, Forsyth, MO (US); Lary D. Andrews, Kansas City, MO (US); Michael Parmely, Kansas City, MO (US); Rohit Medhekar, (US)

Correspondence Address: Thomas R. Stiebel, Jr., Mayer Brown Rowe & Maw, P.O. Box 2828, Chicago, IL, 60690-2828, US

(21) Appl. No.: 10/188,230

(22) Filed: Jul. 1, 2002

Related U.S. Application Data
(63) Continuation-in-part of application No. 09/373,329, filed on Aug. 12, 1999, now Pat. No. 6,413,512.
Application No. 09/373,329 is a continuation-in-part of application No. 09/023,847, filed on Feb. 13, 1998, now abandoned.

(51) Int. Cl. A61K 38/48

(52) U.S. Cl. 424/94.65; 424/94.63

(57) ABSTRACT

Methods compositions and combinations for treating various diseases and conditions that are induced by cytokine activation and response, such as sepsis are provided. The method comprises orally administering a therapeutically effective amount of protease to a mammal to increase the inactivation of cytokines, such as TNF-alpha and INF-gamma. The composition comprises any pharmaceutically acceptable protease, for example, plant and microbial proteases may be used. The composition may be given, for example, singly or in combination with vitamins, minerals, antioxidants, bioflavonoids proanthocyanidins, herbs, herbal extracts, plant and animal concentrates, and non-prescriptive analgesics.

### RELATED APPLICATION

This application claims priority to and is a continuation-in-part of application Ser. No. 09/373,329, filed on Aug. 12, 1999, which was a continuation-in-part of application Ser. No. 09/023,847, filed on Feb. 13, 1998. These two applications are hereby incorporated herein by reference in their entirety to the extent permitted by law.

### FIELD OF THE INVENTION

The present invention relates generally to a method of treating human and animal disease utilizing proteolytic enzymes of plant or microbial origin (“proteases” or “proteinases”) and, more particularly, to a method of modifying the development or manifestation of conditions affected by the action of biologically-active molecules which may be specifically bound or stimulated by activated alpha-2-macroglobulins. Such biologically-active molecules include, but are not limited to, cytokines and other signaling molecules, such as tumor necrosis factor-alpha (TNF-α or TNF-alpha), interferon-gamma (IFN-gamma), leptin, beta-amyloid (βA) and transforming growth factors (TGF).

### BACKGROUND OF THE INVENTION

In many diseases and injuries there is a marked increase in proinflammatory cytokines in the blood. The increased cytokines are believed to contribute to the pathology of the condition. Infection, cancer and tissue injury trigger the production of cytokines. These hormone-like peptides can enter the bloodstream to alter the physiology of distant tissues, or they may behave as paracrine mediators that act only locally. In some disease or injury states, cytokines are beneficial to the host but, in others, cytokines cause the most striking manifestations of the disease (e.g., shock, tissue injury and weight loss). K. J. Tracey, et al., Tumor Necrosis Factor: A Pleiotropic Cytokine and Therapeutic Target, ANN. REV. MED. 45: 491-503 (1994). Proteases and cytokines are intimately interrelated in the body in that cytokines are involved in regulating the production of proteases (Westermarck, J. & Kahari, V. M., Regulation of Matrix Metalloproteinase Expression in Tumor Invasion, FASEB J. 13: 781-92 (1999)), and proteases are frequently involved in the liberation of soluble cytokines. Excess circulating proteases also play an important role in the manifestation of disease or injury.

Alpha2-macroglobulin has a high molecular weight (Mrₕᵤₘₐₙ=725,000 Da), and is composed of identical subunits joined by disulfide bonds. Feldman et. al., Model of alpha₂-Macroglobulin Structure and Function, PROC. NAT'L. ACAD. SCI. USA, 82: 5700-5704 (Sept. 985). The major source of plasma α₂M is the liver (hepatocytes), although other cells including macrophages synthesize and secrete α₂M. This may explain the presence of α₂M in interstitial sites and malignant tissues. LaMarre, et al., Cytokine Binding and Clearance Properties of Proteinase-Activated α₂-Macroglobulins, LAB. INVESTIGATION 65(1): 3-14 (1991). α₂M inhibits proteinases of all four (4) catalytic classes. Barrett, et al., The Interaction of α₂-Macroglobulin With Proteinases: Characteristics and Specificity of the Reaction, and a Hypothesis Concerning its Molecular Mechanism, BIOCHEM. J. 133: 709-724 (1973); Harpel, et al., Studies on Human Plasma α₂-Macroglobulin Enzyme Interactions, J. EXP. MED. 138: 508-521 (1973); Salveson, et al., Covalent Binding of Proteases in Their Reaction With α₂-Macroglobulin, BIOCHEM. J. 187: 695-701 (1980). Proteinases inhibited by α₂M include the coagulation proteinases thrombin and Factor Xa, the fibrinolytic enzymes urokinase-type and tissue-type plasminogen activators, as well as plasmin, kallikrein of the contact system, the neutrophilic proteinases elastase, cathepsin G and collagenase, and several bacterial proteinases. DeBoer, et al., Alpha-2-Macroglobulin Functions as an Inhibitor of Fibrinolytic, Clotting, and Neutrophilic Proteinases in Sepsis: Studies Using a Baboon Model, INFECTION AND IMMUNITY 61(12): 5035-5043 (1993).

Because of the biological importance of activated alpha-2-macroglobulins scavenging of proteinases, several laboratories have attempted to elucidate the structure and function of alpha-2-macroglobulins. The trap mechanism and structure identified by Feldman, et al. has been generally accepted. According to the “trap” hypothesis, proteinases cleave specific peptide bonds within the “bait” amino acids sequence of the alpha-2-macroglobulins subunits and become entrapped within the interior of the alpha-2-macroglobulins as a result of its conformational change. Feldman et. al., supra at 5701. Methylamine mimics the proteinase-induced conformational changes by reacting with the alpha-2-macroglobulins thiol ester bonds, and has therefore been commonly used in studies of activated alpha-2-macroglobulins

A substantial body of research has established that activated alpha-2-macroglobulins complexes acquire a high affinity for binding proinflammatory cytokines, resulting in their removal from the body along with the protease bond in the activated alpha-2-macroglobulins complex. Activated alpha-2-macroglobulins has been shown to play a pivotal role in regulating inflammatory and homeostatic mechanisms of disease and injury by binding major mediators such as tumor necrosis factor-alpha (TNF-α), transforming growth factor-β1 (TGF-β1), transforming growth factor-β2 (TFG-β2), basic fibroblast growth factor (bFGF), platelet-derived growth factor (PDGF), nerve growth factor (NGF), interleukin-1β (IL-1β) and interleukin-6 (IL-6). Wolleberg, et al., Binding of Tumor Necrosis Factor Alpha to Activated Forms of Human Plasma Alpha₂Macroglobulin, AMER. J. PATH., 138 (2): 265-272 (1991). Recent research suggests that various proinflammatory cytokines, and especially TNF-α, play a major role in rheumatoid arthritis. M. Feldmann, et al., Role of Cytokines in Rheumatoid Arthritis, ANN. REV. IMMUN., 14: 397-440 (1996).

One of the hallmarks of most disease processes is acute inflammation, which features the release of neutrophil-derived oxidants. It has recently been shown that “oxidation serves as a switch mechanism that down-regulates the progression of acute inflammation by sequestering TNF-alpha, IL-2, and IL-6, while up-regulating the development of tissue repair processes by releasing beta-FGF, beta-NGF, PDGF, and TGF-beta from binding to alpha2M.” Wu, Patel & Pizzo, Oxidized Alpha2-Macroglobulin (alpha2M) Differentially Regulates Receptor Binding by Cytokines/Growth Factors: Implications for Tissue Injury and Repair Mechanisms in Inflammation. J. IMMUNOL. 161: 4356-65 (1998). The conformationally modified or “activated” alpha-2-macroglobulins is rapidly cleared from the circulation as it more readily binds to specific cell-surface receptors in hepatocytes, macrophages, and fibroblasts and undergoes receptor-mediated endocytosis. These cellular receptors rapidly clear alpha-2-macroglobulins-protease and alpha-2-macroglobulins-methylamine complexes from the systemic circulation, primarily in the liver. Native alpha-2-macroglobulins, on the other hand, is not receptor-recognized and has a prolonged half-life in circulation.

Activated alpha-2-macroglobulins is believed to also play a role in the mediation and regulation of leptin. Leptin is a 16 kDa polypeptide consisting of 167 amino acids that is expressed and secreted from adipose tissue under the control of the “obese gene.” Zhang Y, et al., Positional Cloning of the Mouse Obese Gene and its Human Homologue, NATURE, 372:425-432 (1994). Murine studies indicate that leptin acts on the CNS to regulate body weight through the control of appetite and energy expenditure. Pelleymounter M A., et al., Effects of the Obese Gene Product and Weight Regulation in ob/ob Mice, SCIENCE, 269:540-43 (1995). Leptin has also been shown to affect sympathetic nerve activity, insulin resistance, and renal sodium excretion. Haynes W G, et al., Cardiovascular Consequences of Obesity: Role of Leptin, CLIN. EXP. PHARM. PHYSIOL., 25:65 69 (1998). Obesity is associated with increased activity of the sympathetic nervous system and, therefore, there appears to be a causal link between excess leptin and other systemic, and especially cardiovascular, consequences of obesity. See id.

There are a variety of undesirable consequences of obesity, including insulin resistance, dyslipoproteinemia and hypercoagulability, all of which are probably due to increases in circulating TNF-α and leptin. Halle M., et al., Importance of TNF-alpha and Leptin in Obesity and Insulin Resistance: A Hypothesis on the Impact of Physical Exercise, EXERC. IMMUNOL. REV. 4:77-94 (1998). Activated α₂M has been identified as a leptin-binding factor in human plasma. Thus, the binding of leptin to activated α₂M and its rapid clearance by the α₂M receptor can significantly influence the bioavailability of leptin. Birkenmeier G., et al., Human Leptin Forms Complexes with α₂-Macroglobulin Which are Recognized by the α₂-Macroglobulin Receptor/Low Density Lipoprotein Receptor-Related Protein, EUR. J. ENDOCRIN., 139:224-230 (1998).

TNF-α is thought to be a modulator of gene expression in adipocytes and is implicated in the development of insulin resistance and obesity. Thus, the clearance of TNF-α by activated α₂Ms also appears desirable. Fernandez-Real J M, et al., The TNF-alpha Gene NCO I Polymorphism Influences the Relationship Among Insulin resistance, Percent Body Fat, and Increased Serum Leptin Levels, DIABETES 46(9):1468-72 (1997). The Fernandez-Real group found that increasing transcription of TNF-α using a polymorphism on the TNF-α gene increased serum leptin concentrations in a sample of human subjects. Similarly, diet-induced weight loss reduced TNF-α expression and serum leptin levels and improved insulin sensitivity and lipid metabolism. Halle M., et al., Importance of TNF-alpha and Leptin in Obesity and Insulin Resistance: A Hypothesis on the Impact of Physical Exercise, EXERC. IMMUNOL. REV. 4:77-94 (1998). A composition that activates alpha-2-macroglobulins can also be useful for the treatment of non-insulin dependent diabetes and insulin-resistance effect (see Morimoto, et al., LIFE SCIENCE, 61:795-803 (1997)), Crohn's disease (see U.S. Pat. No. 5,656,272) and cachexia (see Tisdale, Wasting and Cancer, J. NUTRITION, 129 (1^{st} Suppl.): 243S-246S.

Activated alpha-2-macroglobulins also affect the clearance of beta-amyloid (βA). Studies show that increased deposition and aggregation of βA is one of the principal neuropathological features of Alzheimer's disease (AD). Selkoe D J, Cell Biology of the Amyloid, β-Protein Precursor and the Mechanism of Alzheimer's Disease, ANN. REV. CELL Biol., 10:373-403 (1994). Amyloid deposits comprise a 39-43 amino acid peptide(s), which is a proteolytic processing product of the amyloid precursor protein (APP) that is expressed by most, if not all, cells. Once formed, the βA peptide oligomerizes and aggregates into insoluble fibrils that are directly toxic to neurons. Pike C. J., et al., In Vitro Aging of β-Amyloid Protein Causes Peptide Aggregation and Neurotoxicity, BRAIN REV., 563:311-314 (1991). It has been found that the circulating, or brain concentration, of βA in patients with AD is greater than normal patients, and it is believed that factors contributing to βA catabolism and/or clearance of βA may contribute to either diffuse (prearnyloid) or neuritic (senile) plaques in the brain. Van Gool D., et al., α₂-Macroglobulin Expression in Neuritic-Type Plaques in Patients with Alzheimer's Disease, NEUROBIOL. AGING, 14:233-37 (1993). Using radiolabeled βA, Du, et al. found that α₂M binds to βA with high affinity. Du Y, et al., α₂-Macroglobulin as a β-Amyloid Peptide-Binding Plasma Protein, J. NEUROCHEM., 69:299-305 (1997). This indicates that βA is cleared from the brain by conjugation with alpha-2-macroglobulins and endocytosis of the βA/alpha-2-macroglobulins complex by low-density lipoprotein receptor-related protein (LRP). Investigations found that α₂M in AD brains were localized to neuritic plaques and that alpha-2-macroglobulins receptors, or LRP, were concentrated in brain areas affected by AD. Strauss S., Detection of Interleukin-6 and α₂-Macroglobulin Immunoreactivity in Cortex and Hippocampus of Alzheimer's Disease, LAB. INVEST., 66:223-230 (1992).

It has also been discovered that activated α₂M directly stimulates macrophages. See Misra & Pizzo, Ligation Of The Alpha2 Signaling Receptor With Receptor-Recognized Forms Of Alpha2-Macroglobulin Initiates Protein And DNA Synthesis In Macrophages: The Effect Of Intracellular Calcium, BIOCHEM. BIOPHYS. ACTA., 1401:121-8 (1998). Such macrophage stimulation can have beneficial effects, such as fighting bacterial infection.

Despite the growing body of research implicating activated alpha-2-macroglobulins as a mediator of various diseases, pharmacotherapy has failed to address modulating alpha-2-macroglobulins to effect treatment of the diseases. In particular, the prior art has failed to focus on modulating alpha-2-macroglobulins with agents that are effective, well-tolerated by most patients, and economical to use. Most current therapy is directed to altering the function of the cytokines themselves; for example, ENBREL, the commercial product based on Le, et al. U.S. Pat. No. 5,698,195, Methods of Treating Rheumatoid Arthritis Using Chimeric Anti-TNF Antibodies issued Dec. 16, 1997, teaches the use of anti-TNF antibodies specific for human tumor necrosis factor-alpha for the treatment of rheumatoid arthritis. However, such treatment, at approximately $220.00 per week of therapy, has the disadvantage of being expensive. It is administered by injection and therefore carries associated risks. Additionally, Mynott U.S. Pat. No. 5,824,305 focuses on a different mechanism and teaches a method of treating diseases mediated by cyclic nucleotide pathways with purified stem bromelain protease.

Activated alpha-2-macroglobulins plays a key role in influencing the availability and activity of various peptides to specific cells and, therefore, in influencing cellular physiology. Consequently, there is a definite need in the art of mammalian therapeutics for a pharmacological agent that increases the activated or “fast” form of alpha-2-macroglobulins to mediate the effects of the above-mentioned cytokines and other signaling molecules, has a low side effect profile, and is economical to use.

### SUMMARY OF THE INVENTION

In accordance with the present invention, it has now been discovered that certain manifestations of disease and injury may be effectively treated with a mixture of proteases of microbial and/or plant origin. Specifically, for example, exogenous proteases are useful for increasing activated alpha-2-macroglobulin in the blood and extravascular tissue. In one embodiment of the present invention, such proteases are administered orally, either singly or in combination with synergistic ingredients, in the form of capsules (hard and soft), tablets (film coated, enteric coated or uncoated), powder or granules (film coated, enteric coated or uncoated) or liquid (solution or suspension), in an amount to produce the desired pharmacological effect, namely objective improvement of the condition of treated patients by positively influencing the cellular physiology.

The protease may be any pharmaceutically acceptable protease, and in one embodiment is of microbial and/or plant origin, given singly or in combination with vitamins, minerals, antioxidants, bioflavonoids, proanthocyanidins, herbs, herbal extracts, plant and animal concentrates, and analgesics. In another embodiment, the microbial protease is administered in a total daily dosage of at least 100,000 HUT (or equivalent biological activity). In still another embodiment the plant protease is administered in a total daily dosage of at least 50,000 PU (or equivalent biological activity).

A composition and method of use thereof for promoting recovery from soft tissue injury is also disclosed. In one embodiment, the orally administered composition contains a mixture of microbial and plant proteases, antioxidant bioflavonoids, proanthocyanidins, vitamins, minerals, plant concentrates, and excipients. The composition can also include an analgesic.

In one embodiment of the present invention the methods, combinations and compositions can be used to treat mammalian disease or injury manifested by cytokines and other signaling molecules by administering to a mammal oral doses of protease to increase activated alpha-2-macroglobulins in the serum, which in turn serves as a biological response modifier for the disease or injury. The present invention can be employed to treat any disease or injury where activated alpha-2-macroglobulins plays a role.

In yet another embodiment of the present invention the methods, combinations and compositions can be used to supply exogenous protease to the circulation to create a “preemptive” increase in activated alpha-2-macroglobulins capable of binding proinflammatory cytokines and thereby interrupting cytokine-induced pathology.

In yet another embodiment of the present invention the methods, combinations and compositions can be used to provide a protease-based composition and method for decreasing the time required for healing of soft tissue injuries resulting from accidents, sports injuries, various surgical procedures and the like.

In yet another embodiment of the present invention the methods, combinations and compositions can be used to treat disease or injury, such as sepsis, with a composition that is easy to administer, economical, and well-tolerated by patients.

### BRIEF DESCRIPTION OF THE DRAWINGS

FIG. 1 is a diagram of the production process used to produce the proteases used in the present invention.

FIG. 2 is three point graphs illustrating the effects of PBS and A. Oryzae Proteases 4.5 and 6.0 on LPS-induced cytokines (IL-12, TNF-alpha and IFN-gamma) with the cytokine concentration on the Y-axis and PBS and Proteases 4.5 and 6.0 on the X-axis; FIG. 2(a) illustrates the effects of PBS and the listed proteases on IL-12; FIG. 2(b) illustrates the effects of PBS and the listed proteases on TNF-alpha; and FIG. 2(c) illustrates the effects of PBS and the listed proteases on IFN-gamma.

FIG. 3 is a line graph illustrating the amount of IFN-gamma on the Y-axis and amount of protease on the X-axis.

FIG. 4 is three line graphs illustrating the amount of nitrite on the Y-axis and the input of IFN-gamma on the X-axis; FIG. 4(a) illustrates the use of Elastase; FIG. 4(b) illustrates the use of Protease 6.0; and FIG. 4(c) illustrates the use of Peptidase.

FIG. 5 is three line graphs illustrating the amount of nitrite on the Y-axis and the input of TNF-alpha on the X-axis; FIG. 5(a) illustrates the use of Elastase; FIG. 5(b) illustrates the use of Protease 6.0; and FIG. 5(c) illustrates the use of Peptidase.

FIG. 6 is a bar graph illustrating the types of proteases on the Y-axis and the amount of TNF-alpha on the X-axis.

FIG. 7 is a line graph illustrating the amount of nitrite on the Y-axis and the input of IFN-gamma on the X-axis.

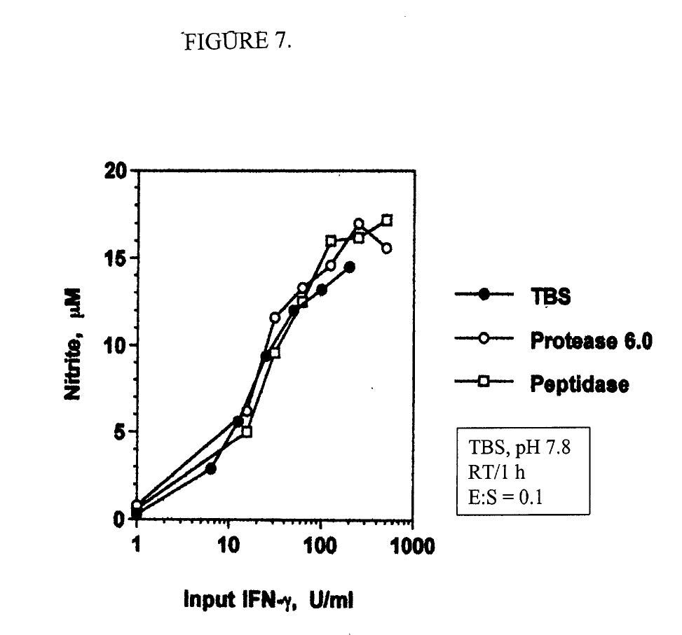

FIG. 8 is a Western blot illustrating the proteolytic effect between protease 6.0 according to the present invention, peptidase and elastase on 100 ng of recombinant mouse IFN-gamma.

### DETAILED DESCRIPTION OF THE INVENTION

Before the present compositions and method of use thereof for increasing activated alpha 2-macroglobulins are disclosed and described, it is to be understood that this invention is not limited to the particular configurations, compositions, process steps and materials disclosed herein, as such configurations, compositions, process steps and materials may vary. It is also to be understood that the terminology employed herein is used for the purpose of describing particular embodiments only and is not intended to be limiting since the scope of the present invention will be limited only by the appended claims and equivalents thereof.

In one embodiment of the invention, a method of treating a disease in a mammal, is provided comprising orally administering to the mammal a pharmaceutical composition comprising two or more proteolytic enzymes from a microbial source selected from the group consisting of Aspergillus oryzae, Aspergillus niger, Aspergillus sojae, Aspergillus flavus, Aspergillus awamori, Pseudomonas and Bacillus subtilis. The proteolytic enzyme is administered in an amount from about 20,000 HUT (or equivalent biological activity) to about 550,000 HUT (or equivalent biological activity) per day. In one embodiment of the present invention the disease treated is selected from the group consisting of sepsis, rheumatoid disease, obesity, cachexia, hypertension, cardiovascular disease, dyslipoproteinemia, insulin resistance, non-insulin dependent diabetes, Crohn's disease, dementia, Alzheimer's disease, infection and soft tissue injury. In yet another embodiment, the composition of the method further comprises a plant protease. In another embodiment the plant protease is administered in an amount of at least about 50,000 PU (or equivalent biological activity) per day. In another embodiment, the composition is administered in an amount from about 100,000 HUT (or equivalent biological activity) to about 350,000 HUT (or equivalent biological activity). In still another embodiment, the composition comprises a plant protease in an amount of at least about 50,000 PU (or equivalent biological activity) per day. In another embodiment, the plant protease comprises bromelain or papain, and mixtures thereof. In another embodiment, composition further comprises a vitamin, a mineral, an antioxidant, a bioflavonoid, a proanthocyanidin, an herb, an herbal extract, a plant concentrate, an animal concentrate or a non-prescription analgesic and mixtures thereof.

In one embodiment of the invention, a pharmaceutical composition is provided, comprising about 20,000 HUT (or equivalent biological activity) to about 550,000 HUT (or equivalent biological activity) of two or more microbial proteases from a microbial source selected from the group consisting of Aspergillus oryzae, Aspergillus niger, Aspergillus sojae, Aspergillus flavus, Aspergillus awamori, Pseudomonas and Bacillus subtilis; and a plant protease in an amount from about 80,000 PU (or equivalent biological activity) to about 1.5 million PU (or equivalent biological activity). In another embodiment the composition further comprises (a) from about 15 mg to 100 mg of ascorbic acid, provided by acceptable salts thereof, and mixtures thereof; (b) from about 15 mg to 100 mg of antioxidant bioflavonoids; or (c) from about 2 mg to 15 mg of proanthocyanidins; and mixtures thereof. In yet another embodiment, composition further comprises (a) from about 5 mg to 150 mg of calcium, provided by acceptable salts thereof, and mixtures thereof; or (b) from about 40 mg to 200 mg of trace mineral-rich algae; and mixtures thereof. In still another embodiment the composition further comprises an effective amount of an analgesic. In yet another embodiment the analgesic comprises acetaminophen, ibuprofen, ketoprofen, salicylates or indomethacin, and mixtures thereof. In another embodiment the analgesic is acetaminophen and said effective amount is from about 65 mg to 500 mg per dosage unit.

In one embodiment of the invention, a method is provided for promoting recovery from soft tissue injury in a patient in need thereof, comprising orally administering to the patient an effective amount of a composition comprising two or more microbial proteases from a microbial source selected from the group consisting of: Aspergillus oryzae, Aspergillus niger, Aspergillus sojae, Aspergillus flavus, Aspergillus awamori, Pseudomonas and Bacillus subtilis, in an amount from about 20,000 HUT (or equivalent biological activity) to about 550,000 HUT (or equivalent biological activity) per day. In another embodiment, the method further comprises a plant protease. In yet another embodiment, the plant protease is administered in an amount of at least about 50,000 PU (or equivalent biological activity) per day.

In one embodiment of the invention, a method for promoting recovery from soft tissue injury is provided comprising orally administering to a patient an effective amount of a composition comprising, per dosage unit, a protease selected from the group consisting of: (a) from about 20,000 HUT (or equivalent biological activity) to 550,000 HUT (or equivalent biological activity) of microbial protease; and (b) from about 80,000 PU (or equivalent biological activity) to 1.5 million PU (or equivalent biological activity) of plant protease; and mixtures thereof. In another embodiment, the composition further comprises: (a) from about 15 mg to 100 mg of ascorbic acid, provided by acceptable salts thereof, and mixtures thereof; (b) from about 15 mg to 100 mg of antioxidant bioflavonoids; or (c) from about 2 mg to 15 mg of proanthocyanidins; and mixtures thereof. In yet another embodiment, the composition further comprises: (a) from about 5 mg to 150 mg of calcium, provided by acceptable salts thereof, and mixtures thereof; or (b) from about 40 mg to 200 mg of trace mineral-rich algae; and mixtures thereof. In another embodiment the composition further comprises an effective amount of an analgesic. In sill another embodiment the analgesic comprises acetaminophen, ibuprofen, ketoprofen or salicylates, indomethacin, and mixtures thereof. In another embodiment the analgesic is acetaminophen and the effective amount is from about 65 mg to 500 mg per dosage unit.

The present invention also contains kits comprising a pharmaceutical composition is provided, comprising about 20,000 HUT (or equivalent biological activity) to about 550,000 HUT (or equivalent biological activity) of two or more microbial proteases from a microbial source selected from the group consisting of Aspergillus oryzae, Aspergillus niger, Aspergillus sojae, Aspergillus flavus, Aspergillus awamori, Pseudomonas and Bacillus subtilis; and a plant protease in an amount from about 80,000 PU (or equivalent biological activity) to about 1.5 million PU (or equivalent biological activity).

It must be noted that, as used in this specification and the appended claims, the singular forms “a,” “an” and “the” include plural referents, unless the context clearly dictates otherwise. Thus, for example, reference to an “antioxidant” includes a mixture of two or more antioxidants, reference to a “vitamin” includes reference to one or more of such vitamins, and reference to a “microbial protease” includes references to two or more of such microbial proteases.

As used herein, “effective amount” means an amount of a composition according to the present invention that is non-toxic but sufficient to provide the selected local or systemic effect and performance at a reasonable benefit-to-risk ratio attending any product of this nature. An effective amount of an analgesic is an amount sufficient to provide a selected level of pain relief.

As used herein, “acceptable salts” means, for example, salts of citric acid or ascorbic acid, wherein such salts are generally regarded as safe for human consumption.

In general, the present invention is directed to a method combination or composition for alleviating the manifestations of mammalian disease or injury by administering an effective amount of a mixture of proteolytic enzymes (proteases) derived from microbial and/or plant origin. The present invention may be used to treat for example any type of cytokine or signaling peptide-mediated disease or injury whereby the proteases serve as high affinity biological response modifiers through their activation of alpha-2-macroglobulins to either enhance the clearance of the particular cytokine or signaling molecule such as TNF-α, leptin, beta-amyloid and interleukins, or to enhance the delivery to a particular tissue of a particular cytokine or signaling molecule including transforming growth factors, basic fibroblast growth factor, platelet derived growth factor, and nerve-growth factor. Diseases and injuries treated by the present invention include rheumatoid disease, excess weight including obesity, cachexia, hypertension and cardiovascular disease, dyslipoproteinemia, insulin resistance, non-insulin dependent diabetes, Crohn's disease, dementia of the Alzheimer's type, infections and soft tissue injuries, including surgery.

Cytokines play a major role in the manifestation of inflammation, which is a predominant biological reaction to a myriad of injurious agents and events. It is well known that host-defensive and reparative processes in inflammation can be inimical to the body's welfare. Common characteristics of inflammation are bruising, swelling and pain. The body's defensive mechanisms can bring about the release of products that are toxic to the host, or destruction of some of its tissues. Additional detrimental consequences of inflammation include fibrin deposition and reduction in vascularity, causing changes in tissue permeability and creating additional morphologic barriers to the penetration of antibodies or pharmacological agents into the injured area. Some of the autolysis products released by tissue necrosis often constitute a good medium for microorganisms and can even antagonize the antimicrobial activity of many pharmaceutical agents, thereby exacerbating the injury and prolonging the recovery process.

Current drug treatment for inflammatory conditions that incorporate steroids and NSAIDs is aimed at complete inhibition of inflammation. The goal of using orally administered proteases in treating soft tissue injury is to minimize, not inhibit, the inflammatory process by accelerating the normal healing process. Undesirable side effects from the use of steroids and NSAIDs dictate a need for safer, but still effective, alternatives for inflammation relief. The method and compositions of this invention are directed to provide this relief without the adverse side effects.

Other measures aimed solely at the etiologic agent will often prove inadequate. On the other hand, a therapeutic agent directed at the host response level and capable of modifying defensive and reparative processes of localized inflammation without depriving the body of its benefits would prove invaluable. In one embodiment of the invention, the method and compositions of this invention rely upon the ability of orally administered proteases to activate alpha-2-macroglobulins to minimize the detrimental effects of proinflammatory cytokines.

In a disease and injury free state, mammals have very little circulating protease in the blood. Circulating alpha-2-macroglobulin, on the other hand, is abundant in human plasma (˜2 ng/ml), and has a major function of non-specifically binding protease. Applicants demonstrate that the exogenous administration of proteases increases the amount of activated α₂M in the blood and extravascular tissue, which enhances the clearance of the cytokine or signaling molecule of interest minimizing its detrimental actions, and thereby having highly desirable therapeutic effects.

A reduction of circulating TNF-α, IFN-gamma, leptin and βA is beneficial to patients, for example, with rheumatoid and cardiovascular diseases, obesity and plaque deposition associated with Alzheimer's disease, respectively. The elevation of activated alpha-2-macroglobulins/TGF-β complexes also improves wound healing by delivering the activated TGF-β where it is most beneficial for tissue repair.

The absorption of orally administered proteases in animals and humans has been extensively studied. The prevailing finding of these studies is that proteases can be partially absorbed intact, with activity preserved, from the digestive tract and subsequently distributed systemically in the blood. This is evidenced by anecdotal observations of the systemic action of oral protease supplementation and also well-controlled studies demonstrating that proteins, including proteolytic enzymes, can be detected in the circulation in active whole form after oral administration. Castell J. V., Friedrich G., Kuhn C. S. & Poppe G. E., Intestinal Absorption Of Undegraded Proteins In Men: Presence Of Bromelain In Plasma After Oral Intake, AM. J. PHYSIOL., 273: G139-G146 (1997).

The discovery of the beneficial effect of protease on modulating biological functions such as inflammation and tissue repair has inspired considerable experimentation into exogenous sources and their clinical effects. The most common source of exogenous proteases used for inflammation is the pancreas of pigs and cows slaughtered for meat, commercially known as pancreatin. Pancreatin contains such proteases as trypsin, chymotrypsin, carboxypeptidase and elastase. Plant sources of protease, primarily bromelain derived from pineapple and papain derived from papaya, are the second most studied exogenous sources of proteases for inflammation. Proteases from microbial sources, such as fungi and bacteria, are relative newcomers as therapeutic agents for inflammation.

Animal, plant and microbial proteases have been found to differ significantly in their molecular weight, molecular configuration, substrate specificity, kinetic reactions, pH and temperature reaction optima, inhibitors, cofactors and composition of the proteolytic active site. S. Schwimmer, Source Book for Food Enzymology, p. 89-122 (1981). For example, animal enzymes optimally digest food in the more alkaline pH of the small intestines whereas microbial enzymes are stable and even active in a lower pH. Such differences in functional properties do not support the assumption that all proteolytic enzymes will share common properties in terms of systemic absorption, distribution and physiological action.

Early in the study of proteases, it was observed that the administration of animal-derived proteases could accelerate the healing of an inflamed site. Therefore, a large database exists of clinical results from orally administered, animal-derived proteases establishing the effectiveness of these proteases as therapeutic agents for inflammatory conditions. However, a clear mechanism of physiological action for animal-derived proteases is yet to be determined. The same is true for plant-derived proteases. Plant proteases have been found to have a positive effect on inflammation but only one mechanism of action for bromelain has been proposed and supported by research. See U.S. Pat. No. 5,824,305. The largest body of evidence supporting the use of protease for inflammatory conditions studied the effects of a mixture of papain, bromelain, trypsin, chymotrypsin, pancreatin and rutin. In most cases, the mixture was in addition to standard medical care. It must also be noted that a large quantity of the mixture was required to observe beneficial results, sometimes as many as 30 tablets per day.

Microbial proteases have been extensively used over the past 40 years in the food industry to improve the taste, texture or solubility of certain foods but their effectiveness as mediators of disease and injury is the focus of this invention. The combination of microbial proteases with plant proteases and other synergistic ingredients in this invention serve to improve the effectiveness of the inventive compositions.

The broad range of physiological action and greater biological activity per gram of microbial proteases present in the invention means only a few capsules are required for the desired clinical effect compared to the large dosage required for animal- and plant-derived protease products. Because of the high potency provided by microbial enzymes, the amount of plant proteases in this invention can be reduced significantly compared to known compositions. The invention does not contain animal-derived products, and thus is acceptable to patients who may object to the ingestion of animal products.

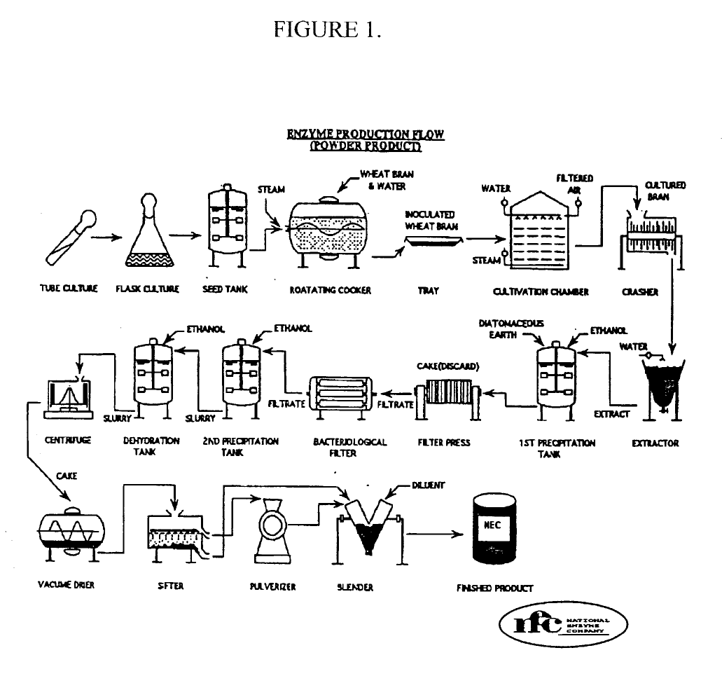

Since three goals of the present invention are ease of administration, low cost to the user and minimal side effects, in one embodiment of the present invention the composition and method employed to obtain the proteases is relatively economical and results in pure (hypoallergenic), highly concentrated preparations. An exemplary method for producing microbial proteases from various species of Aspergillus fungi is solid state fermentation illustrated diagrammatically in FIG. 1, although other production methods known in the art are also acceptable. Referring to FIG. 1, the fermentation process begins by taking a population of the desired fungi from a test tube culture and transferring it to a large flask for additional growth. This cultured fungi is then moved to a seed tank where it is further propagated. The resulting concentrated suspension of fungi is then transferred to a rotating cooker and mixed with sterilized koji (wheat or rice bran), water and steam where it is cooked for a sufficient period of time to inoculate the koji with fungi. The inoculated koji is then moved onto large trays, which are then transported to a cultivation chamber where the fungi are permitted to grow. Fermentation under controlled temperatures and humidity conditions may take from a few days to a week or more to complete.

At the conclusion of fermentation, the cultured koji is then transferred to a crusher device, which pulverizes the koji mash. The resulting pulverized mash is then moved to an extractor to filter the particulate matter from the slurry. For some processes, there may also be microfiltration or ultrafiltration steps to concentrate the aqueous enzymes before precipitation. The slurry is then moved to a first precipitation tank where it is mixed with ethanol and filtered through diatomaceous earth and then run through a filter press where the cake is discarded. The filtrate from the filter press is then processed through a bacteriological filter before it is moved to a second precipitation tank for further filtering and precipitation. The ethanol precipitation and bacteriological filter steps produce enzymes that are microbially very “clean,” for example, they have very low microbes when compared to other food products such as fluid or pasteurized milk. The slurry is then centrifuged and the resulting cake is transferred to a vacuum dryer for drying. The dried proteinaceous material is then passed through a sifter and then a pulverizer to reduce the particle size. This material is then placed in a blender and diluent may or may not be added to standardize the potency of the finished powder product.

In one embodiment of the present invention, the proteolytic enzymes are administered orally in daily dosages in the form of a pharmaceutical composition comprising mixtures of about at least 100,000 HUT (or equivalent biological activity) of microbial proteases, and/or mixtures of about 50,000 PU (or equivalent biological activity) of plant proteases.

One embodiment of the composition of the invention employs the dosage unit designations HUT (Hemoglobin Unit Tyrosine) and PU (Papain Units), which are Food Chemical Codex activity units to describe the preferred potencies of the proteolytic enzymes. One skilled in the art will recognize that there are many different activity unit designations used for microbial and plant proteases depending upon the type of application and geographic location of the enzyme supplier. However, regardless of the activity unit designation employed, equivalent biological activity can be determined by readily available laboratory analysis.

In one embodiment of the present invention the dose is intended to be administered on an empty stomach (for example, at least two hours after a meal or snack or one hour before a meal or snack). In yet another embodiment the dose administered to a mammal, particularly a human is sufficient to effect a therapeutic response in a reasonable timeframe. The dose and timing of the dose will be determined by the strength of the particular composition administered and by the condition of the person, as well as the body weight of the person to be treated. The size of the dose also will be determined by the existence, nature and extent of any adverse side effects that might accompany the administration of a particular composition. The formulations described herein may be administered concurrently with other necessary medications. For example, they may be administered together with non-steroidal anti-inflammatory agents or the more recent cyclooxygenase-2 (COX-2) inhibitors in the treatment of arthritis.

The composition of the present invention may also include additional ingredients such as other enzymes, vitamins, minerals, antioxidants, bioflavonoids, proanthocyanidins, herbs, herbal extracts, plant and animal concentrates and analgesics. In one embodiment, the composition additionally comprises a pharmaceutically acceptable carrier. Pharmaceutically acceptable carriers are also well known to those who are skilled in the art. The choice of carrier will be determined, in part, both by the particular composition and by the particular method used to administer the composition. One skilled in the art will appreciate that suitable methods of administering the protease-based formulations of the present invention to a mammal are available and, although more than one method can be used to administer a particular composition, a particular method can provide a more immediate and more effective reaction than another. There is a wide variety of suitable forms of administration of the pharmaceutical compositions of the present invention.

In one embodiment of the present invention the forms of administration are oral forms known in the art of pharmaceutics. The pharmaceutical compositions of the present invention may be orally administered as a capsule (hard or soft), tablet (film coated, enteric coated or uncoated), powder or granules (coated or uncoated) or liquid (solution or suspension). The formulations may be conveniently prepared by any of the methods well-known in the art. The pharmaceutical compositions of the present invention may include, for example, one or more suitable production aids or excipients including fillers, binders, disintegrants, lubricants, diluents, flow agents, buffering agents, moistening agents, preservatives, colorants, sweeteners, flavors and pharmaceutically compatible carriers.

In one embodiment of the present invention a composition comprising a mixture of proteases from microbial and plant sources combined with vitamin C, bioflavonoids, proanthocyanidins, calcium and mineral-rich algae is provided. In one embodiment, the microbial protease composition is comprised of a mixture of proteolytic enzymes from Aspergillus oryzae, Aspergillus niger, Aspergillus sojae, Aspergillus flavus, Aspergillus awamori, Pseudomonas or Bacillus subtilis. The use of microbial proteases in this formulation as anti-inflammatory agents and promoters of wound healing constitutes an improvement upon known compositions for these purposes. The microbial portion of this composition is comprised of three fungal proteases derived from strains of Aspergillus and one bacterial protease derived from Bacillus subtilis. All of these microbial proteases are commercially derived either by the methodology previously described or by other published methods; see, e.g., S. Schwimmer, Source Book of Food Enzymology, p. 89-122 Avi Publishing, 1981, and provide a wide range of enzymatic activities. The predominant characteristics of the microbial protease mixture is a pH optimum for activity in the range of 3.0 to 8.0, which corresponds generally to the physiological pH of 7.4, and broad substrate specificity, with a slight preference for cleaving the carboxyl side of hydrophobic amino acid residues. In addition, one of the microbial proteases provides endopeptidase activity and exopeptidase activity observed as preferential cleavage of leucine residues from the amino terminus of peptides. Another microbial protease is advantageous for its stability at a pH as low as 2.0.

In one embodiment of the present invention, the microbial proteases used in the present composition are, for example, complementary in that they provide overlap in pH optima, substrate specificity and mode of cleaving the peptide backbone of proteins. Unlike many other types of proteins, these proteases are more stable in the presence of stomach acid and, therefore, are less likely to be destroyed by the process of digestion. The proteolytic activity of human serum has been observed to increase after ingestion of proteasecontaining preparations, indicating gastric survival of proteases. Of the total quantity of enzymes ingested, approximately 10% to 40% are absorbed into the general circulation. The molecular weight of microbial protease is about 35 kDa. The broad range of physiological conditions under which these proteases remain active is necessary, since microenvironmental conditions within the body may include pH values much different than 7.4, the norm for human serum.

In yet another embodiment of the present invention the mixture of proteases in the embodiments include plant-derived proteases, such as bromelain and papain. Bromelain is the collective name for the proteolytic enzyme composition derived from the stem of the pineapple plant, Ananas comosus. Papain is purified from the fruit of the tropical melon tree, Carica papaya. The primary use of highly concentrated microbial proteases in the present embodiment has allowed a relatively small portion of the composition to be comprised of plant proteases. This is notable because the plant sources of both bromelain and papain are subject to environmental influences, which can have a significant impact upon the commercial availability and price of both plant enzymes.

Bromelain and papain are referred to as thiol proteases and contain a cysteine residue at the active site. Under oxidizing conditions, such as inflammation, the thiol group of this cysteine loses a hydrogen atom and may crosslink with another thiol group, forming a disulfide bridge or, alternatively, cross-linking with another residue through the same oxidative process. In this oxidized state, the bromelain and papain lose activity. The present composition solves this problem, for example, through the inclusion of antioxidant vitamin C, bioflavonoids and proanthocyanidins that prevent the oxidation of the active sulfhydryl group of the thiol proteases.

Bromelain and papain have been studied more extensively than the microbial enzymes and possess potent anti-inflammatory properties. The known proteolytic enzymes of bromelain and papain share a high degree of amino acid sequence homology around the active center, and evidence suggests that bromelain and papain use the same catalytic mechanism. Bromelain differs from papain, however, in having a different specificity of cleavage. In addition, the known proteolytic enzymes of bromelain are glycoproteins, whereas papain is a simple protein. See U.S. Pat. No. 5,767,066; Taussig et. al., J. ETHNOPHARMACOL., 22:191-203 (1988). Research has established that bromelain is absorbed in an intact, functional state into human circulation. Castel, et al., Intestinal Absorption of Undegraded Proteins in Men: Presence of Bromelain in Plasma After Oral Intake, AM. J. PHYSIOL., 273:139-46 (1977). The molecular weight of bromelain is approximately 24-26 kDa.

Microbial proteases have different active sites and generally are not labile under oxidative conditions. By focusing on microbial proteases in one embodiments of the invention, the stability of the proteolytic activity of the composition is greatly improved thus providing superiority over known compositions that relied solely or primarily upon the actions of the plant thiol proteases, bromelain or papain.

Vitamin C (ascorbic acid), a bioessential organic acid, is included in one embodiment because it is well known as an antioxidant, is essential in the biosynthesis of collagen, and for supporting immune function, thereby improving the healing of damaged tissues. In one embodiment of the present invention vitamin C is provided in the composition in the form of calcium ascorbate, which is a calcium salt of ascorbic acid. In-another embodiment, it may also contain, for example, other potent antioxidants in the form of bioflavonoids and proanthocyanidins. The bioflavonoid, quercetin (3.3′,4′,5, 7-pentahydroxyflavone), has been shown to inhibit certain mediators of the inflammatory process, and another bioflavonoid, rutin (3-rhamnoglucoside of 5,7,3′,4′-tetrahydroxyflavonol), has been shown to reduce chemically induced inflammation in clinical studies. Research has shown that the active antioxidants in grape extract, known as proanthocyanidins, are anti-inflammatory agents capable of inhibiting the process of edema.

In another embodiment of the invention the composition includes calcium and trace minerals for use in homeostatic functions, in musculoskeletal healing, as electrolytes and as enzyme cofactors. Calcium is provided in one example of an embodiment as calcium citrate and calcium ascorbate because the mineral is optimally absorbed by the body from these two forms. Clinical experience with high doses of orally administered microbial protease has resulted in muscle cramping in some patients. The alpha-2-macroglobulins-protease complexes have been shown to cause a release of intracellular calcium (Misra et al., BIOCHEM. J. 15:885-91 (1993)), which is a cause of cramping. Oral administration of calcium in conjunction with microbial protease can alleviate the cramping. The trace minerals in this exemplary embodiment are provided by mineral-rich algae such as marine kelp, Laminaria sp., and Irish moss, Chondrus crispus.

One example of an embodiment may also contain an effective amount of a non-prescription or prescription analgesics, such as acetaminophen, ibuprofen, ketoprofen, salicylates such as acetylsalicylic acid (aspirin) and indomethacin. Dosages of such analgesics should not exceed federal regulations. In accordance therewith, an exemplary dosage range for acetaminophen is from about 65 mg. to 500 mg. per dosage unit.

The composition of the another embodiment is orally administered in capsule (hard or soft), tablet (coated or uncoated), powder or granule (coated or uncoated) or liquid (solution or suspension) form and dissolves easily in the stomach. One skilled in the art will perceive other physical forms of the composition that will be equally as useful such as enteric-coated or other sustained release dosage forms. The composition may optionally include, for example, production aids or excipients. In an example of an embodiment, the composition is prepared by blending together the stated raw materials in an agglomerator so as to result in a product having a uniform composition with the proportions of the components as indicated. The agglomerated material is then placed in gelatin or other capsules, pressed into tablets, dissolved or suspended in liquid or packaged in a suitable container.

The formula, per dosage unit, of one embodiment is as follows:

| | |
|---|---|
| Microbial Proteases | 20,000 HUT to 550,000 HUT |
| | (or biological equivalents) |
| Plant Proteases | 80,000 PU to 1.5 million PU |
| | (or biological equivalents) |
| Ascorbic Acid from Calcium Ascorbate | 15 mg. to 100 mg. |
| Bioflavonoids (Rutin and Quercetin) | 15 mg. to 100 mg. |
| Proanthocyanidins (Grape Extract) | 2 mg. to 15 mg. |
| Calcium (from Calcium Citrate and | 5 mg. to 150 mg. |
| Calcium Ascorbate) | |
| Trace Mineral-Rich Algae | 40 mg. to 200 mg. |

In one embodiment of the present invention a dose is administered on an empty stomach (at least, for example, two hours after a meal or snack and one hour before a meal or snack) five times per day. A dose will be determined by the strength of the particular composition administered and by the condition of the person, as well as the body weight of the person to be treated. The size of a dose also will be determined by the existence, nature and extent of any adverse side effects that might accompany the administration of this composition. Additionally, dosage forms comprising only proteases in the ranges noted above may be employed.

The present invention will be further illustrated by the following examples that are not intended to limit the scope of the invention.

### EXAMPLE 1

Groups of CF1 mice will be injected intraperitoneally (i.p.) with preformed protease-alpha-2-macroglobulin complexes, preformed methylamine-alpha-2-macroglobulin complexes or bovine serum albumin only. Fifteen minutes later doses of either recombinant tumor necrosis factor-alpha or interferon gamma will be injected i.p. It is hypothesized that a significant reduction in circulating cytokine levels will be observed in the mice that receive protease and methylamine-activated alpha-2-macroglobulins relative to the placebo.

### EXAMPLE 2

Adult CFI mice will be challenged with bacterial endotoxic lipopolysaccharides (LPS) to induce a rapid increase in circulating levels of acute phase proteins and cytokines, including tumor necrosis factor-alpha and interferon-gamma. Mice challenged with LPS will be injected with protease, preformed protease-alpha 2-macroglobulin complexes, preformed methylamine-alpha 2-macroglobulin complexes, or vehicle only. It is postulated that significant reductions in circulating cytokine levels will be observed in all three treatment groups compared to the control group.

### EXAMPLE 3

Two females were admitted for treatment of varicose veins. Treatment was to consist of injection of sodium morrhuate, a sclerosing agent, into the affected veins of each leg. One leg was to be treated at each visit with the visits separated by one month. This particular procedure lent itself well for study, as one leg could be used as the control (normal post-operative treatment) and the other as the dependent variable (normal post-operative treatment plus the protease-based composition). Follow-up visits were made at one, two and four weeks after each procedure. The first leg was treated with standard medical care. At the week-one follow-up examination, the injected vessels were observed to be clotted and palpable. The more superficial vessels contained dark blue clots. Upon palpation of the vessels, a noticeable amount of pain was reported by both patients. At two weeks post-procedure, tissue bruising extended up to 3 mm on either side of the sclerosed vessel. Skin discoloration had changed from dark blue to yellow-green. The larger sclerosed vessels were still palpable but no longer painful. At four weeks post-procedure, bruising was apparent only in the largest vessels. The surrounding skin had a brown appearance that was due to staining by hemosiderin, a breakdown product of blood hemoglobin. The second leg was treated in a similar manner with the exception that the protease-based composition according to the present invention was added to the treatment regimen. The patients took the composition immediately after the procedure and four days following. The week-one post-procedure examination revealed bruising in only 50% of the vessels treated, and complete clearance of clots from 25% of the smaller superficial vessels. Pain was present over the largest vessels only upon deep palpation. Two weeks after treatment, 80% of all bruising and discoloration had resolved, and in the remaining areas only yellow-green bruising was observed. At four weeks after treatment, all bruising was resolved and no brown hemosiderin staining was noted. Both patients stated that they had fewer subjective problems following the second procedure.

### EXAMPLE 4

A 35-year-old male underwent lipoplasty of the right and left flank. Approximately 400 cc were aspirated from each flank. Observations on day 3 post-lipoplasty revealed significant bruising of the flanks extending to the genitalia. Edema was noted throughout the area and the patient was unable to rest without analgesia. On day 10 little change was noted in bruising and edema, but discomfort had diminished. Twenty days post-lipoplasty, minimal yellowing of the skin was observed with pain and edema resolved. Twelve weeks later the patient underwent a 475-cc aspiration of the lower abdomen. In addition to normal post-operative management, the protease-based composition according to the present invention was added to the treatment regiment. Discomfort from the procedure was present only on the first night following surgery. Day 10 examination showed complete resolution of bruising and minimal levels of edema. Examination on day 20 showed complete resolution of all symptoms.

### EXAMPLE 5

A 39-year-old female underwent lipoplasty of the lower abdomen, from which approximately 900 cc was aspirated. On days 3 and 10 post-surgery, severe bruising, edema and discomfort were noted. Analgesics were required for three weeks following the surgery, at the end of which discomfort was minimal but still present. Ten weeks following the first surgery, an additional lipoplasty was performed with 1700 cc removed from the flanks. Post-operative care protease-based composition according to the present invention. Examination at 3 days showed moderate bruising and edema with severe discomfort level. On day 10 complete resolution of bruising was noted while edema and pain levels were unchanged. Complete resolution of pain was noted by the 20-day post-operative examination with edema present only on deep palpation.

### EXAMPLE 6

A 38-year-old female underwent lipoplasty with 1000 cc aspirated from the lower abdomen. Day 3 revealed significant bruising, edema and discomfort. On day 10 bruising and edema were reduced to moderate levels while discomfort remained unchanged. Some residual bruising and edema were present on day 20, while the discomfort had resolved. Twelve weeks later the patient had 1200 cc aspirated from her flanks. The protease-based composition according to the present invention was added to the normal treatment protocol. Examination on day 3 showed low to moderate bruising and edema. Any discomfort had completely resolved. The day 20 exam revealed resolution of bruising and discomfort while edema was noted only on deep palpation.

### EXAMPLE 7

In this example, the protease-based composition according to the present invention was used in a double-blind study of 41 patients undergoing plastic surgery (lipoplasty). Eighteen patients received standard post-operative treatment whereas 23 patients received standard post-operative treatment plus the protease-based composition according to the present invention. All patients were assessed at specified post-operative intervals for ecchymosis (bruising), edema, and pain. The length of time required to resolve all three conditions was significantly reduced in the treatment group receiving the protease-based composition (Lomax, J. E., The Use of Oral Proteolytic Enzymes in the Post-lipoplasty Patient, LIPOPLASTY 15: 10-15 (1998)).

### EXAMPLE 8

In this example, two proteases (formulations “4.5” (about 500,000 or about 600,000 HUT) and “6.0” (about 500,000 HUT) produced by Aspergillus oryzae were used to produce an anti-inflammatory effect upon mice injected with bacterial endotoxic lipopolysaccharide (LPS). Protease 4.5 and 6.0 are blends of acid, neutral, and alkaline exo- and endopeptidases. The numbers 4.5 and 6.0 represent about the optimum pH for each protease blend. A. oryzae protease 4.5 and 6.0 were found to protect mice against the proinflammatory effects of the bacterial LPS. The enzymes partially and selectively degrade the cytokine IFN-gamma in vitro. However, it is significant that protease 6.0 augmented the action of another microbial protease, Pseudomonas elastase, leading to greater inactivation of biological activity of IFN-gamma. However, because plasma levels of both IFN-gamma and TNF-alpha were decreased in Aspergillus protease-treated, LPS-challenged mice (compared to mice only challenged with LPS), it is hypothesized that these fungal proteases behave differently in vivo than in vitro. Flow cytometry was used to enumerate cytokine-producing cells. However, it failed to detect significant effects of these proteases on the biosynthesis of these two cytokines in endotoxin-challenged mice, thus it was hypothesized that the effect was explained by proteolysis and not biosynthesis of cytokines.

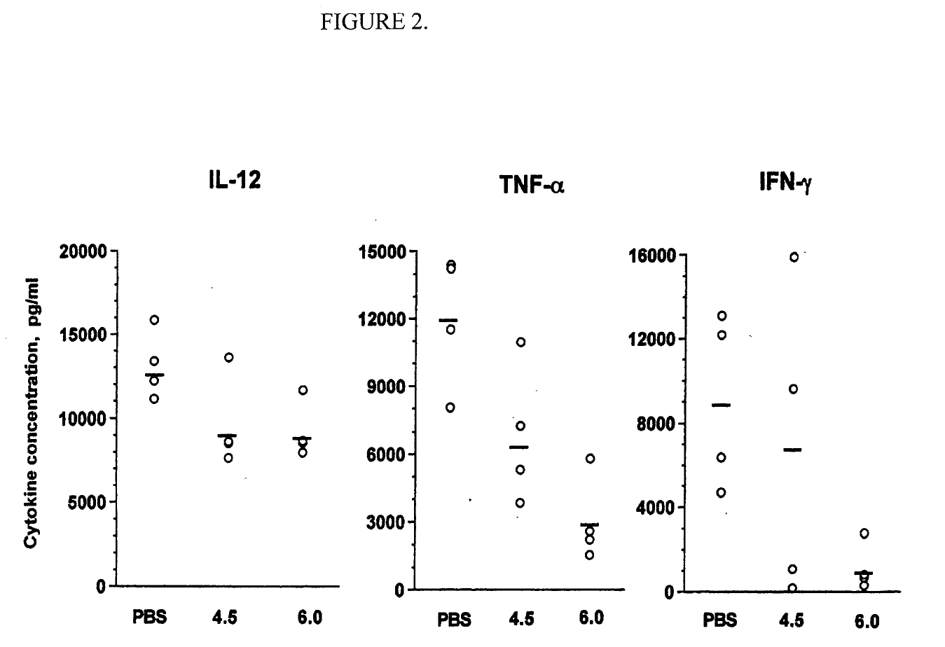

The effects of Aspergillus protease 4.5 or 6.0 on proinflammatory cytokines were measured by determining to what extent cytokine production induced by bacterial endotoxic LPS was altered in protease-treated mice. Animals first received an intraperitoneal injection with phosphate-buffered saline (PBS) or protease 4.5 or 6.0 at a dose of 3 mg/kg body weight. They were challenged 30 minutes later with intraperitoneal injection of 100 micrograms E. coli LPS. Sera was collected 1 hour for TNF-alpha or 6 hours for IL-12 and IFN-gamma and cytokine concentrations were measured by ELISA. Each data point in FIG. 2 represents an individual mouse. The results shown in FIG. 2 indicate that IL-12 levels were unaffected by protease treatment, but TNF-alpha and IFN-gamma responses were significantly decreased in mice that had been treated with protease 6.0 30 minutes prior to LPS challenge. Pretreatment with protease 4.5 led to a more variable effect on serum IFN-gamma concentrations. On the basis of these data, protease 6.0 was selected for further characterization, and A. oryzae peptidase, which lacked activity in the endotoxicosus model (see below), was chosen as a negative control enzyme.

### EXAMPLE 9

While not wishing to be bound by theory, it is believed that Protease 6.0 is modifying IFN-gamma following its secretion from lymphocytes. In this test, it was determined whether the enzyme degraded or inactivated the cytokine in vitro. Recombinant mouse IFN-gamma was treated with Aspergillus oryzae protease 6.0, peptidase or Pseudomonas aeruginosa elastase for 4 hours at 37 C.° at various enzyme-to-substrate mass ratios (0.003-0. 1). Elastase was selected as a control in this experiment based on previously published data indicating that this enzyme degrades and inactivates a number of human and mouse cytokines in vitro. One hundred (100) ng mouse IFN-gamma was incubated in 100 mM Tris buffer; 0.25% bovine serum albumin, pH 7.5 in the presence of increasing mass enzyme-to-substrate ratios of Pseudomonas elastase, Aspergillus protease 6.0 or Aspergillus peptidase. The enzyme to-substrate ratios used were 0.004, 0.01, 0.03, 0.1. After 4 hours of incubation at 37 C.°, the samples were boiled in SDS sample buffer and analyzed by Western blotting with a polyclonal anti-IFN-gamma antibody. The results indicated that significant differences existed between the effects of elastase and either protease 6.0 or peptidase. While elastase produced a number of IFN-gamma fragments that differed significantly in M from the native IFN-gamma, Aspergillus protease 6.0 only partially hydrolyzed IFN-gamma yielding what appeared to be a stable large peptide product. Aspergillus peptidase at low concentrations produced a similar partial hydrolysis, but degraded the IFN-gamma more extensively at higher enzyme to substrate (E:S) ratios.

### EXAMPLE 10

A similar analysis to Example 9 was then performed with a constant enzyme-to-substrate ratio but varying the time of incubation. One hundred (100) ng IFN-gamma was incubated for 1, 2, 3 or 4 hours as described in Example 9 with Aspergillus protease 6.0, peptidase and Pseudomonous elastase at an enzyme-to-substrate ratio of 0.033. Then the samples were boiled and analyzed by Western blotting with anti-IFN-gamma. Elastase hydrolyzed IFN-gamma extensively, whereas the two Aspergillus proteases 6.0 and peptidase yielded stable peptides that were only slightly smaller than the native IFN-gamma. This data suggests that only a single or limited number of cleavage sites on IFN-gamma are attacked by the Aspergillus enzymes under these conditions, whereas elastase is capable of cleaving peptide bonds at multiple sites.

### EXAMPLE 11

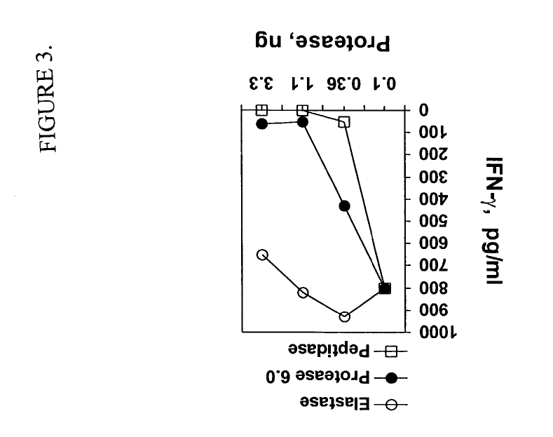

An analysis of the effects of the Pseudomonas and Aspergillus proteases on the antigenicity of IFN-gamma yielded evidence similar to Example 10 for differences in substrate specificity as illustrated in FIG. 3. One hundred (100) ng IFN-gamma was incubated at 37 C.° for 24 hours in 100 mM Tris buffer; 0.25% BSA, having a pH of 7.5 with the enzyme-to-substrate quantities of 0.004, 0.01, 0.03 and 0.1 for the three enzymes. The reaction mixtures were then diluted in PBS containing 10% fetal bovine serum (FBS) to stop the reactions and immediately added to ELISA plates for measurement of residual IFN-gamma. The sandwich ELISA used for this purpose contained two monoclonal antibodies, one serving as a “capture antibody” and the other as an enzyme-conjugated “detection antibody.” Thus, alteration of either the capture epitope or the detection epitope of IFN-gamma would result in a significant decrease in antigenicity. Pseudomonas elastase tested over a range of concentrations did not significantly decrease IFN-gamma detection in the ELISA. By contrast, the Aspergillus protease 6.0 and peptidase produced a nearly complete loss of native antigenicity. Thus, the antigenic determinants recognized by this pair of monoclonal antibodies were not affected by the Pseudomonas elastase, but were destroyed by both Aspergillus protease 6.0 and peptidase.

### EXAMPLE 12

The mouse C2C12 skeletal myoblast cell line can be stimulated to produce large amounts of nitric oxide by combinations of IFN-gamma and TNF-alpha. By providing one of these cytokines in excess, the activity of the second cytokine unknown sample can be measured (for example, by measuring nitric oxide (NO) concentrations in the culture fluids). Nitrite is rapidly oxidized to nitrites in tissue culture, which can be easily measured by a colorimetric assay using the Griess reagent.

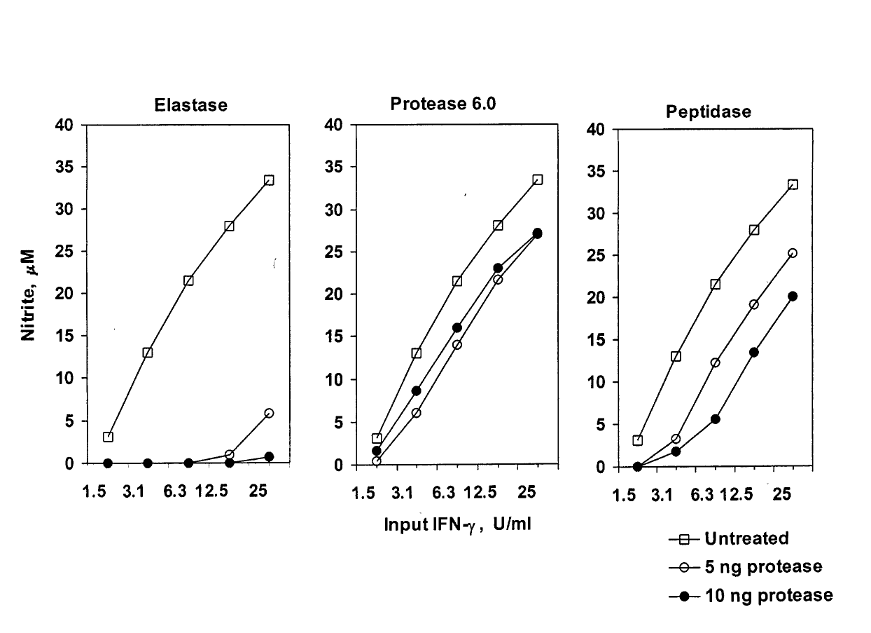

Aliquots of IFN-gamma were incubated in Tris buffer at 37 C.° for 24 hours or treated with two 5 ng and 10 ng concentrations of Aspergillus protease 6.0, peptidase and Pseudomonas elastase. Then the reaction mixtures were diluted in culture medium containing 10% FBS to inhibit enzyme activity and added to C2C12 cell cultures together with excess recombinant TNF-alpha. Twenty four (24) hours later, culture fluids were recovered and nitrite concentrations were measured. Pseudomonas elastase inactivated mouse IFN-gamma, Aspergillus protease 6.0 had an insignificant effect on the biological activity of IFN-gamma in this assay. Likewise, Aspergillus peptidase decreased activity of the cytokine only slightly (for example, less than 50%). See FIG. 4

### EXAMPLE 13

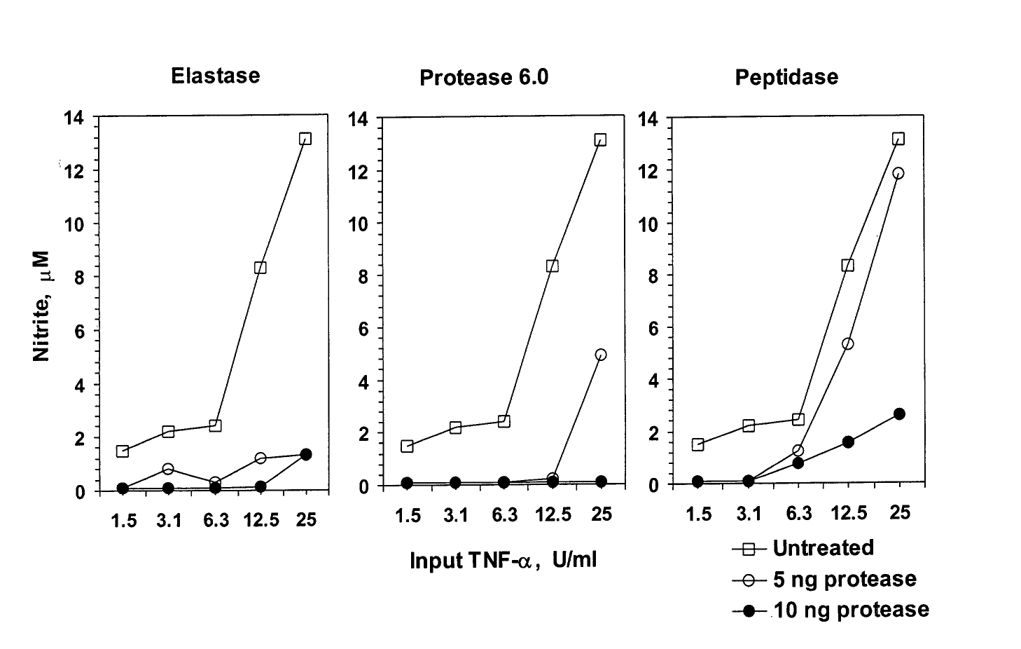

In this example, Aspergillus proteases 6.0 (5 ng) and peptidase (5 ng) contribution to IFN-gamma inactivation was studied. Elastase (5 ng) was combined with either protease 6.0, peptidase or elastase and mixed with 100 ng IFN-gamma. Incubation conditions followed those described in Example 12. Residual IFN-gamma bioactivity was assessed 24 hours later by measuring the ability of the IFN-gamma mixtures to induce nitrite production by TNF-alpha-stimulated C2C12 cells. The results are shown in FIG. 5 and indicate that both Aspergillus protease 6.0 and peptidase augmented the inactivation of IFN-gamma caused by Pseudomonas elastase.

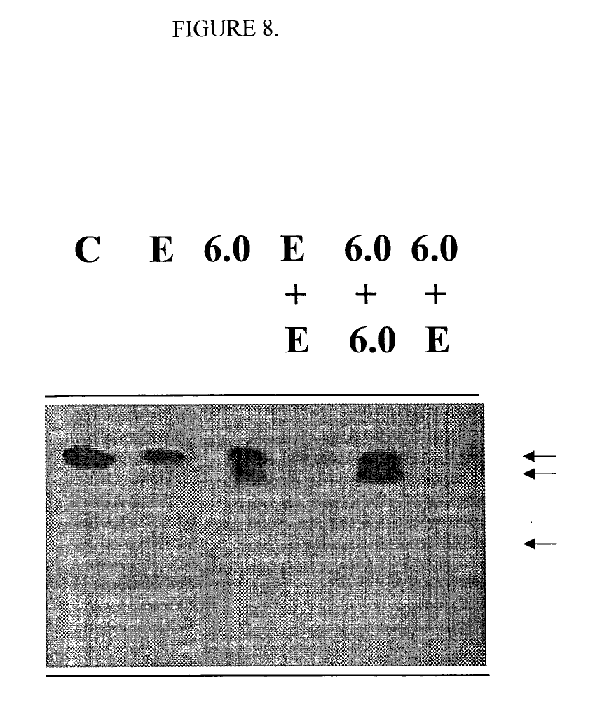

This synergistic proteolytic effect between the Aspergillus proteases 6.0, peptidase and Pseudomonas elastase was also seen on Western blots (FIG. 8). One hundred (100) ng recombinant mouse IFN-gamma was incubated in 100 mM Tris buffer; 0.25% BSA, pH 7.5 for 4 hours at 37 C.° with 5 ng of Pseudomonas elastase or Aspergillus protease 6.0 or peptidase. The reactions were stopped by the addition of FBS-containing PBS, and the samples were analyzed by Western blotting for evidence of cleavage. Thus, combinations of suboptimal concentrations of either protease 6.0 or peptidase with elastase led to nearly complete proteolysis of IFN-gamma similar to that obtained with a double concentration of elastase alone.

### EXAMPLE 14

TNF-alpha is a potent mediator of systemic inflammatory responses to pathogenic microbes and microbial products, including endotoxic LPS. Mice that were treated with A. oryzae protease 4.5 or 6.0 and then challenged with LPS showed variably decreased levels of serum TNF-alpha (FIG. 2).

To evaluate potential protease effects on the biosynthesis of TNF-alpha, mice in three groups were treated with either saline or one of the proteases and then challenged with LPS. CF1 mice were injected with saline or one of the proteases intraperitoneally in the amount of 3 mg/kg body weight and then challenged with 100 micrograms LPS 30 minutes later. Six hours later their livers were collected and hepatic macrophages were prepared by collagenase digestion and Percoll gradient centrifugation. The cells were then stained with fluorochrome-conjugated antibody to mouse TNF-alpha or nonspecific rat IgG as a negative control. (Table 1). The protease treatment did not alter the frequency or intensity of staining of TNF-alpha-expressing cells, suggesting that the number of cells synthesizing the TNF-alpha was essentially equivalent in the three treatment groups.

TABLE 1
Flow cytometric analysis of the effects of Aspergillus protease 6.0
and peptidase on TNF-alpha biosynthesis in vivo.

| | % Hepatic Macrophages | |
|---|---|---|
| | Expressing Intracellular TNF- | Mean Fluorescence |
| Pretreatment | alpha | Intensity |
| PBS | 1.06 | 10.72 |
| Protease 6.0 | 0.77 | 10.35 |
| Peptidase | 0.90 | 14.00 |

CF1 mice were injected with saline or the proteases i.p. (3 mg/kg body weight) and then challenged with 100 micrograms LPS 30 min later. Six hours later their livers were collected and hepatic macrophages were prepared by collagenase digestion and Percoll gradient centrifugation. The cells were then stained with fluorochrome-conjugated antibody to mouse TNF-alpha or nonspecific rat IgG as a negative control.

To determine whether or not the Aspergillus proteases 6.0 and peptidase cleaved mouse TNF-alpha, Western blotting was performed on incubation mixtures containing TNF-alpha and one of the three proteases, Aspergillus 6.0, peptidase and elastase. Fifty (50) ng aliquots of recombinant TNF-alpha were mixed with 12 ng of each protease in 150 mM Tris buffer, pH 7.8 and incubated at 37 C.° for the indicated times. Then the mixtures were boiled in sample buffer, electrophoresed and probed with anti-TNF-alpha. While elastase cleaved TNF-alpha within 4 hours and eliminated its detection of the parent polypeptide by Western blotting, neither of the Aspergillus proteases 6.0 or peptidase altered TNF-alpha structure in this manner.

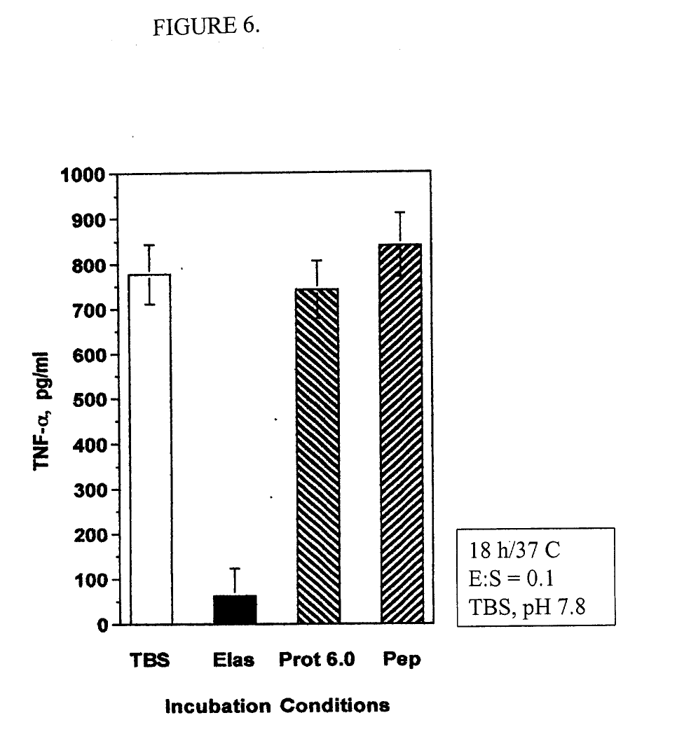

A similar finding was made when TNF-alpha antigenicity was evaluated in the enzyme-linked inmmunoassay, as illustrated in FIG. 6. Two hundred (200) ng aliquots of TNF-alpha were incubated at 37 C.° in 150 mM Tris buffer, pH 7.8 with or without 24 ng of one of the three microbial proteases. After 18 hours, the reaction mixtures were diluted in FBS-containing PBS to stop the reactions and immediately added to ELISA plates for measurement of residual TNF-alpha. Here too, treatment of mouse TNF-alpha for 18 hours at 37 C.° with Pseudomonas elastase decreased antigenicity by over 90%. By contrast, neither of the Aspergillus enzymes 6.0 or peptidase altered the ability of TNF-alpha to interact with the monoclonal antibodies used in this sandwich ELISA. Because antigenicity of proteins is highly conformation-dependent, it seemed unlikely that either Aspergillus protease had significantly altered the native structure of the protein cytokine.

### EXAMPLE 15

In this example, the administration of the Aspergillus proteases 4.5, 6.0 and peptidase was tested to determine the affect the biological activity of TNF-alpha in vivo. Mice were injected intraperitoneally with either PBS or one of the proteases in the amount of 3 mg/kg body weight. Thirty minutes later they were challenged with 20 mg D-galactosamine plus either 100 ng E. coli O111:B4 LPS or 3 micrograms recombinant mouse TNF-alpha. Lethality was assessed over the subsequent 24 hours. Pooling the results of the three experiments indicated a significant difference between protease 4.5-treated and PBS-treated mice at p<0.02 (Chi Square); the protease 6.0-treated group was also significantly different from PBS control group (p<0.02; Chi Square). The results are found in Table 2.

TABLE 2
Aspergillus proteases protect mice from the proinflammatory
effects of endotoxic LPS.

| | | | Lethality |
|---|---|---|---|
| Experiment | Pretreatment | Challenge | (Deaths/Total) |
| 1 | PBS | LPS + D-galNH₂ | 7/8 |
| | Protease 4.5 | LPS + D-galNH₂ | 4/8* |
| 2 | PBS | LPS + D-galNH₂ | 8/8 |
| | Protease 4.5 | LPS + D-galNH₂ | 4/8* |
| 3 | PBS | LPS + D-galNH₂ | 7/8 |
| | Protease 4.5 | LPS + D-galNH₂ | 5/8* |
| | Protease 6.0 | LPS + D-galNH₂ | 4/8* |
| | Peptidase | LPS + D-galNH₂ | 7/8 |
| 4 | PBS | TNF-α + D-galNH₂ | 8/8 |
| | Protease 4/5 | TNF-α + D-galNH₂ | 8/8 |
| | Protease 6.0 | TNF-α + D-galNH₂ | 8/8 |

Mice were injected i.p. with either PBS or the indicated protease (3 mg/kg body weight). Thirty min later they were challenged with 20 mg D-galactosamine plus either 100 ng E. coli O111:B4 LPS or 3 micrograms recombinant mouse TNF-alpha. Lethality was assessed over the subsequent 24 hours.
*Pooling the results of the three experiments indicated a significant difference between protease 4.5-treated and PBS-treated mice at p < 0.02 (Chi Square); the protease 6.0-treated group was also significantly different from PBS control group (p < 0.02; Chi Square)

TABLE 3
Summary of the effects of Pseudomonas and
Aspergillus proteases on mouse IFN-gamma and TNF-alpha

| | | Effects of: | | | Table |
|---|---|---|---|---|---|
| | | Pseudomonas | Aspergillus | Aspergillus | or |
| Cytokine | Property | Elastase | Protease 6.0 | Peptidase | FIG. |
| IFN- | Antigenicity | — | [symbol] | [symbol] | |
| gamma | (ELISA) | | | | |
| | Biological activity | | | | |
| | in vitro | | | | |
| | (single enzyme) | [symbol] | — | [symbol] | |
| | (with elastase) | [symbol] | [symbol] | [symbol] | |
| | Biosynthesis in | N.D.* | — | — | |
| | vivo | | | | |
| | (flow cyotometry) | | | | |
| TNF- | Proteolysis | [symbol] | — | — | |
| alpha | (Western blot) | | | | |
| | Antigenicity | [symbol] | — | — | |
| | Biological activity | — | — | — | |
| | in vitro | | | | |
| | Biological activity | N.D. | — | — | Table 2 |
| | in vivo | | | | |
| | Biosynthesis in | N.D. | — | — | Table 1 |
| | vivo | | | | |
| | (flow cyotometry) | | | | |

*N.D. = not determined [symbol]
= decreased; [symbol] = decreased to a greater extent compared to other treatments; see specific data

The results illustrate that the invention can inactivate cytokines and their precursors, thus having a therapeutic effect on diseases induced by cytokine activity, such as sepsis. The amount of protease necessary can be experimentally determined. It is understood, however, that specific dose levels of the therapeutic agents of the present invention for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. It is understood in the art that studies employing rodents, such as rats, can be applied to humans. Further, it is well known in the art that the specific examples illustrated above can be applied to human oral dosage. Typically, dosage-effect relationships from in vitro and/or in vivo tests initially can provide useful guidance on the proper doses for subject administration. Studies in animal models, such as rodents, generally may be used for guidance regarding effective dosages for treatment of sepsis and other diseases or injuries. In terms of treatment protocols, it should be appreciated that the dosage to be administered will depend on several factors, including the particular agent that is administered, the route administered the condition of the particular subject, etc. Generally speaking, one will desire to administer an amount of the agent that is effective to achieve a serum level commensurate with the concentrations found to be effective in vitro. Thus, where an agent is found to demonstrate in vitro activity at, for example, 10 ng/ml, one will desire to administer an amount of the agent that is effective to provide about a 10 ng/ml concentration in vivo. Determination of these parameters is well within the skill of the art. These considerations, as well as effective formulations and administration procedures are well known in the art and are described in standard textbooks.

While the invention has been described with reference to preferred embodiments, it will be understood by those of ordinary skill in the art that the invention is not limited thereto. It is obvious that variations in the preferred methods of the present invention may be used and that it is intended that the invention may be practiced otherwise than as specifically described herein. Accordingly, this invention includes all modifications or equivalents encompassed within the spirit and scope of the invention as defined by the appended claims. All publications cited to or referenced herein are incorporated by reference in their entirety.

What is claimed is:

1. A method of treating a disease in a mammal in need thereof, comprising: orally administering to the mammal a pharmaceutical composition comprising two or more proteolytic enzymes from a microbial source selected from the group consisting of Aspergillus oryzae, Aspergillus niger, Aspergillus sojae, Aspergillus flavus, Aspergillus awamori, Pseudomonas and Bacillus subtilis, in an amount from about 20,000 HUT (or equivalent biological activity) to about 550,000 HUT (or equivalent biological activity) per day; wherein the disease is selected from the group consisting of sepsis, rheumatoid disease, obesity, cachexia, hypertension, cardiovascular disease, dyslipoproteinemia, insulin resistance, non-insulin dependent diabetes, Crohn's disease, dementia, Alzheimer's disease, infection and soft tissue injury.

2. The method of claim 1, wherein the composition further comprises a plant protease.

3. The method of claim 2, wherein the plant protease is administered in an amount of at least about 50,000 PU (or equivalent biological activity) per day.

4. The method of claim 2, wherein the composition comprises a plant protease in an amount of at least about 50,000 PU (or equivalent biological activity).

5. The method of claim 2, wherein the plant protease comprises bromelain or papain, and mixtures thereof.

6. The method of claim 1, wherein the composition is administered in an amount from about 100,000 HUT (or equivalent biological activity) to about 350,000 HUT (or equivalent biological activity) per day.

7. The method of claim 1, wherein the composition further comprises at least one of a vitamin, a mineral, an antioxidant, a bioflavonoid, a proanthocyanidin, an herb, an herbal extract, a plant concentrate, an animal concentrate or a non-prescription analgesic, and mixtures thereof.

8. A pharmaceutical composition, comprising: about 20,000 HUT (or equivalent biological activity) to about 550,000 HUT (or equivalent biological activity) of two or more microbial proteases from a microbial source selected from the group consisting of Aspergillus oryzae, Aspergillus niger, Aspergillus sojae, Aspergillus flavus, Aspergillus awamori, Pseudomonas and Bacillus subtilis; and a plant protease in an amount from about 80,000 PU (or equivalent biological activity) to about 1.5 million PU (or equivalent biological activity).

9. The composition of claim 8, further comprising: (a) from about 15 mg to 100 mg of ascorbic acid, provided by acceptable salts thereof, and mixtures thereof; (b) from about 15 mg to 100 mg of antioxidant bioflavonoids; or (c) from about 2 mg to 15 mg of proanthocyanidins; and mixtures thereof.

10. The composition of claim 8, further comprising ingredients: (a) from about 5 mg to 150 mg of calcium, provided by acceptable salts thereof, and mixtures thereof; or (b) from about 40 mg to 200 mg of trace mineral-rich algae; and mixtures thereof.

11. The composition of claim 8, further comprising an effective amount of an analgesic.

12. The composition of claim 11, wherein said analgesic comprises: acetaminophen, ibuprofen, ketoprofen, salicylates and indomethacin, and mixtures thereof.

13. The composition of claim 11, wherein the analgesic is acetaminophen and the effective amount is from about 65 mg to 500 mg per dosage unit.

14. A method for promoting recovery from soft tissue injury in a patient in need thereof, comprising: orally administering to the patient an effective amount of a composition comprising two or more microbial proteases from a microbial source selected from the group consisting of: Aspergillus oryzae, Aspergillus niger, Aspergillus sojae, Aspergillus flavus, Aspergillus awamori, Pseudomonas and Bacillus subtilis, in an amount from about 20,000 HUT (or equivalent biological activity) to about 550,000 HUT (or equivalent biological activity) per day.

15. The method of claim 14, further comprising: a plant protease.

16. The method of claim 15, wherein the plant protease is administered in an amount of at least about 50,000 PU (or equivalent biological activity) per day.

17. A method for promoting recovery from soft tissue injury comprising orally administering to a patient an effective amount of a composition comprising, per dosage unit, a protease selected from the group consisting of: (a) from about 20,000 HUT (or equivalent biological activity) to 550,000 HUT (or equivalent biological activity) of microbial protease; and (b) from about 80,000 PU (or equivalent biological activity) to 1.5 million PU (or equivalent biological activity) of plant protease; and mixtures thereof.

18. The method of claim 17, wherein said composition further comprises: (a) from about 15 mg to 100 mg of ascorbic acid, provided by acceptable salts thereof, and mixtures thereof; (b) from about 15 mg to 100 mg of antioxidant bioflavonoids; or (c) from about 2 mg to 15 mg of proanthocyanidins; and mixtures thereof.

19. The method of claim 17, wherein said composition further comprises: (a) from about 5 mg to 150 mg of calcium, provided by acceptable salts thereof, and mixtures thereof; or (b) from about 40 mg to 200 mg of trace mineral-rich algae; and mixtures thereof.

20. The method of claim 17, wherein the composition further comprises an effective amount of an analgesic.

21. The method of claim 20, wherein the comprises: acetaminophen, ibuprofen, ketoprofen, salicylates or indomethacin, and mixtures thereof.

22. The method of claim 20, wherein the analgesic is acetaminophen and the effective amount is from about 65 mg to 500 mg per dosage unit.
